(19) United States
(12) Patent Application Publication
(10) Pub. No.: US 2008/0147135 A1
(43) Pub. Date: Jun. 19, 2008

(54) FILTER CIRCUIT WITH VARIABLE CAPACITANCE FOR USE WITH IMPLANTABLE MEDICAL DEVICES

(76) Inventor: Scott A. Hareland, Lino Lakes, MN (US)

Correspondence Address: MEDTRONIC, INC., 710 MEDTRONIC PARKWAY NE, MINNEAPOLIS, MN, 55432-9924, US

(21) Appl. No.: 11/612,940

(22) Filed: Dec. 19, 2006

(51) Int. Cl. A61N 1/36; A61N 1/39; H03J 5/02

(52) U.S. Cl. 607/7; 607/30; 331/36 R

(57) ABSTRACT

An implantable medical device (IMD) as described herein includes an input filter circuit having variable capacitor elements that can be electronically adjusted based upon current operating conditions (such as electromagnetic conditions, noise conditions, and/or environmental conditions). The variable capacitor elements can be adjusted to accommodate pre-designated operating modes of the IMD and/or dynamically in response to changing operating conditions. In one embodiment, the variable capacitor elements are realized using digitally programmable switched capacitor arrangements.

### TECHNICAL FIELD

The subject matter described herein relates generally to implantable medical devices (IMDs). More particularly, the subject matter described herein relates to the use of variable capacitance elements with a filter circuit of an IMD.

### BACKGROUND

IMDs are used to treat patients suffering from a variety of conditions. IMDs can be utilized in a variety of applications, such as drug or fluid delivery, monitors, and therapeutic devices for other areas of medicine, including metabolism, endocrinology, hematology, neurology, muscular disorders, gastroenterology, urology, ophthalmology, otolaryngology, orthopedics, and similar medical subspecialties.

Examples of IMDs involving cardiac devices are implantable pacemakers and implantable cardioverter-defibrillators (ICDs). Such electronic medical devices generally monitor the electrical activity of the heart and provide electrical stimulation to one or more of the heart chambers when necessary. For example, pacemakers are designed to sense arrhythmias, i.e., disturbances in heart rhythm, and, in turn, provide appropriate electrical stimulation pulses at a controlled rate to selected chambers of the heart in order to correct the arrhythmias and restore the proper heart rhythm. The types of arrhythmias that may be detected and corrected by IMDs include bradycardias (unusually slow heart rates) and certain tachycardias (unusually fast heart rates).

ICDs also detect arrhythmias and provide appropriate electrical stimulation pulses to selected chambers of the heart to correct the abnormal heart rate. In contrast to pacemakers, however, an ICD can also provide pulses that are much stronger and less frequent, where such pulses are generally designed to correct fibrillation, which is a rapid, unsynchronized quivering of one or more heart chambers, and severe tachycardias, during which the heartbeats are very fast but coordinated. To correct such arrhythmias, ICDs deliver low, moderate, or high-energy therapy pulses to the heart.

Many IMDs are configured to receive leads that detect electrical signals from the body and/or deliver electrical therapy to the body. Incoming signals are usually fed to a filter circuit having capacitors; the filter circuit enables the IMD to effectively capture the desired electrical sensor signals while also preventing unwanted electrical noise from contaminating the input signals. For example, capacitive elements are used to isolate the relatively low magnitude signals derived from body-generated electrical activity. The size of the capacitor(s) determines the filtering characteristics of the filter circuit. Conventional IMDs employ fixed value capacitors on the inputs of the IMD sensing circuit. These fixed value capacitors must be sized such that the IMD is capable of operating effectively under all foreseeable medical and environmental conditions. However, fixed capacitors can degrade the quality of the captured electrical signals when the IMD is used in certain environments (e.g., when the patient undergoes magnetic resonance imaging or certain surgical procedures that expose the patient to electromagnetic energy, or when the patient is exposed to unusually high levels of RF energy).

### BRIEF DESCRIPTION OF THE DRAWINGS

Aspects and features of the present invention will be appreciated as the same becomes better understood by reference to the following detailed description of the embodiments of the invention when considered in connection with the accompanying drawings, wherein:

FIG. 1 is an illustration of an IMD in the body of a patient;

FIG. 2 is a schematic representation of an embodiment of an IMD;

FIG. 3 is a schematic representation of an embodiment of an IMD having a filter circuit with variable capacitor elements;

FIG. 4 is a schematic diagram of an embodiment of a filter circuit having variable capacitor elements;

FIG. 5 is a schematic diagram of an embodiment of a variable capacitor element;

FIG. 6 is a schematic diagram of another embodiment of a variable capacitor element; and

FIG. 7 is a flow chart that illustrates an embodiment of an IMD filter adjustment process.

### DETAILED DESCRIPTION

A filter circuit as described herein is suitable for use in an IMD. The filter circuit includes variable capacitor elements that can be electrically adjusted or programmed as necessary to enhance the quality of the received sensor signals. The use of variable capacitors in this manner allows the IMD to dynamically adjust itself in response to current electromagnetic conditions in the environment. Variable capacitors may also be utilized to support different operating modes for an IMD that contemplate designated operating environments, such as a normal mode, an MRI mode, or the like.

As used herein, a “node” means any internal or external reference point, connection point, junction, signal line, conductive element, or the like, at which a given signal, logic level, voltage, data pattern, current, or quantity is present. Furthermore, two or more nodes may be realized by one physical element (and two or more signals can be multiplexed, modulated, or otherwise distinguished even though received or output at a common mode).

The embodiments described herein can be implemented in any IMD that is configured to process electrical signals that might be susceptible to unwanted electromagnetic interference or noise. For example, such electrical signals may be sensor signals received by the IMD via one or more leads connected to the IMD. At present, a wide variety of IMDs are commercially available or proposed for clinical implantation. Such IMDs include pacemakers as well as ICDs, drug delivery pumps, cardiomyostimulators, cardiac and other physiologic monitors, nerve and muscle stimulators, deep brain stimulators, cochlear implants, and artificial organs (e.g., artificial hearts). In addition, as the technology advances, it is contemplated that IMDs shall become even more complex with respect to programmable operating modes, menus of operating parameters, and monitoring capabilities of increasing varieties of physiologic conditions and electrical signals. It is to be appreciated that embodiments of the subject matter described herein will be applicable in such emerging IMD technology as well.

In contrast to conventional IMDs, an IMD configured as described herein employs an input filter circuit having one or more variable capacitor elements (rather than fixed capacitors). The variable capacitor elements are electrically adjustable or programmable to enable adjustment of the node capacitance on the IMD inputs in response to control signals generated by the IMD itself. The total IMD input capacitance can be changed depending upon pre-programmed settings and/or dynamically in response to operating or environmental conditions detected by the IMD. This functionality results in a robust IMD design that can operate effectively in a variety of clinical and other environments that might otherwise introduce an undesirable amount of noise into the input signals. This functionality may also be utilized to provide for dynamic tradeoffs in noise immunity versus signal quality in different IMD operating environments.

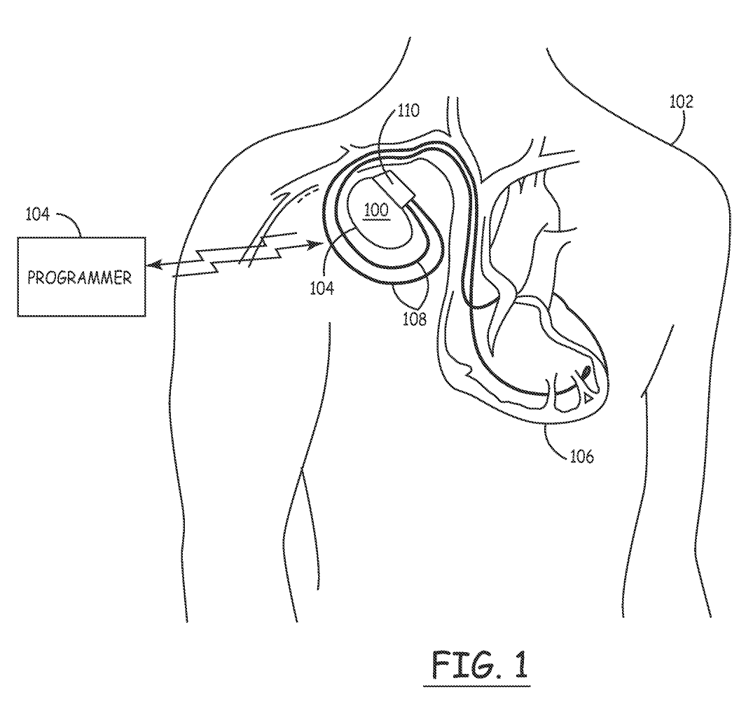

FIG. 1 is an illustration of an IMD 100 implanted in the body of a patient 102. FIG. 1 also depicts an external communication device (such as a programmer 104 for IMD 100) that is not implanted within patient 102. In certain embodiments, telemetry communications can take place between IMD 100 and programmer 104 using known wireless telemetry techniques and technologies. The arrows in FIG. 1 represent such telemetry communications. In practice, a given communication session between programmer 104 and IMD 100 may be unidirectional or bidirectional (in this example, FIG. 1 depicts bidirectional communications).

In certain embodiments, when IMD 100 is used for cardiac applications (e.g., to provide cardiac sensing, pacing, and/or defibrillation functions for patient 102), IMD 100 can be a cardiac device—for example, a pacemaker, an ICD, a hemodynamic monitor, or the like. As described above, however, IMD 100 is not limited to such applications or such devices. In this example, IMD 100 is implanted beneath the skin or muscle of patient 102 and IMD 100 is typically oriented to the skin surface. When IMD 100 is used for cardiac applications (as shown in FIG. 1), IMD 100 is electrically coupled to the heart 106 of the patient 102 through pace/sense or cardioversion/defibrillation electrodes operatively coupled to lead conductor(s) of one or more endocardial leads 108. In this example, leads 108 are coupled to a connector block 110 of IMD 100 in a manner well known in the art.

As generally mentioned above, among other design functions, programmer 104 is suitably designed for non-invasive communication with IMD 100, where such communication is enabled via downlink and uplink communication channels. Generally, any form of portable programmer, interrogator, recorder, monitor, or telemetered signals transmitter and/or receiver found suitable for communicating with IMD 100 could be used for programmer 104. As described in more detail below, programmer 104 may be suitably configured to control the switching of operating modes and/or variable capacitor settings for IMD 100.

In certain embodiments, programming commands or patient data can be transmitted between IMD 100 and programmer 104. Telemetry communications may, for example, utilize a high frequency signal (or UHF, or VHF signal). In practice, the telemetered data can be encoded in any of a wide variety of telemetry formats. While not being limited to such, some examples of particular data encoding or modulation types and/or techniques that can be utilized with such data transmissions include noise modulation, general spread spectrum encoding, bi-phase encoding, quadrature phase shift keying, frequency shift keying (FSK), time division multiple access (TDMA), frequency division multiple access (FDMA), pre-emphasis/de-emphasis of baseband, vestigial, code division multiple access (CDMA), quadrature amplitude modulation (QAM), pi/8, quad-QAM, 256-QAM, 16-QAM, delta modulation, phase shift keying (PSK), quadrature phase shift keying (QPSK), quadrature amplitude shift keying (QASK), minimum shift keying, tamed frequency modulation (TFM), orthogonal frequency division multiplexing (OFDM), Bluetooth, any 802.11 modulation configuration, worldwide interoperability for microwave access (WiMAX), any 802.16 modulation configuration, 802.15.4, and Zigbee. Note that the “mode” used by the transceivers may be selected to optimize performance based on implant depth input and QoS input.

In certain embodiments, the uplink and downlink telemetry transmissions between IMD 100 and programmer 104 follow a telemetry protocol that formulates, transmits, and demodulates data packets each comprising a bit stream of modulated data bits. In certain embodiments, the data packets are formulated of a data bit stream with a preamble, data and error checking data bits.

Programmer 104 may be suitably configured to function as a programming device that provides data, programming instructions, and other information to an IMD having variable capacitor elements as described herein. Moreover, programmer 104 may be suitably configured to control the switching of operating modes of the IMD, where different operating modes correspond to different settings for one or more variable capacitor elements. An embodiment of programmer 104 includes a processing unit (not visibly shown in FIG. 1). As should be appreciated, the processing unit can include any of a wide variety of devices. While not being limited to such, the processing unit, in certain embodiments, can be a personal computer type motherboard, e.g., a computer motherboard including a microprocessor and related circuitry such as digital memory. The processing unit is suitably configured to support the features and operations of programmer 104 described herein.

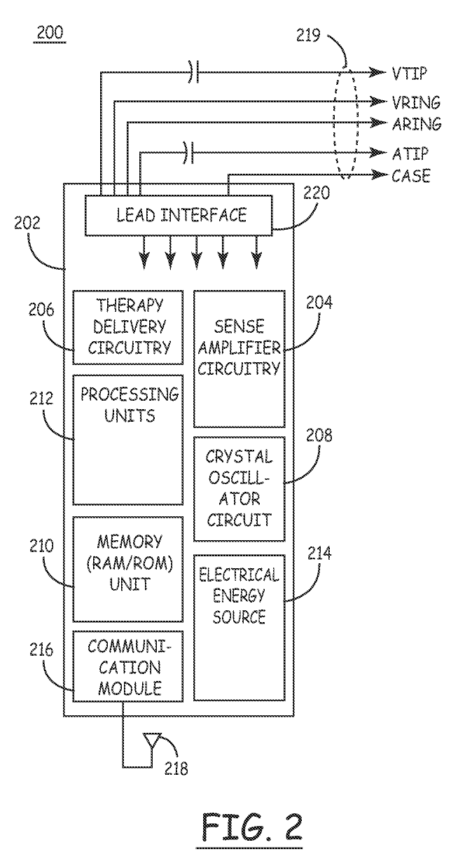

FIG. 2 is a schematic representation of an embodiment of an IMD 200 that can be configured to implement the adaptive filtering feature described herein. IMD 100 and/or any other IMD implanted in patient 102 may be configured as shown in FIG. 2. As can be seen from FIG. 2, IMD 200 includes primary circuitry 202 for managing the operation and function of IMD 200, with such primary circuitry 202 being contained within a hermetic enclosure of IMD 200. In one embodiment, the enclosure is realized as an electrically conductive housing for the internal components of IMD 200. The primary circuitry 202 includes a number of electrical components, such as, without limitation: sense amplifier circuitry 204; therapy delivery circuitry 206; a crystal oscillator circuit 208; a suitable amount of memory 210, which may include random-access memory (RAM) and/or read-only memory (ROM); a processing unit 212; and an electrical energy source 214. In certain embodiments, the primary circuitry 202 also includes a communication module 216 and one or more antennas 218 configured to enable IMD 200 to communicate with other devices. It should be appreciated that the below descriptions of the primary circuitry 202 within the IMD 200 are merely exemplary configurations.

In certain embodiments, when IMD 200 is used for cardiac applications (e.g., to provide cardiac sensing and pacing functions for the patient), the IMD 200 is coupled to one or more endocardial leads 219 which, when implanted, extend transvenously between the implant site of the IMD 200 and the patient's heart, as previously noted with reference to FIG. 1. As mentioned above, the physical connections between the leads 219 and the various internal components of IMD 200 are facilitated by means of a conventional connector block assembly. Electrically, the coupling of the conductors of the leads 219 and internal electrical components of IMD 200 may be facilitated by means of a lead interface circuit 220 which functions, in a multiplexer-like manner, to selectively and dynamically establish necessary connections between various conductors in the leads 219 and individual electrical components of the IMD 200, as would be familiar to those of ordinary skill in the art. In certain embodiments, with respect to such cardiac applications, the various conductors in the leads 219 can include atrial tip and ring electrode conductors, ATIP and ARING, and ventricular tip and ring electrode conductors, VTIP and VRING. For the sake of clarity, the specific connections between the leads 219 and the various components of the IMD 200 are not shown in FIG. 2, although such connections will be familiar to those of ordinary skill in the art. For example, in cardiac applications, the leads 219 will necessarily be coupled, either directly or indirectly, to the sense amplifier circuitry 204 and the therapy delivery circuitry 206, in accordance with common practice, such that cardiac electrical signals may be conveyed to the sense amplifier circuitry 204 and such that stimulating pulses may be delivered by the therapy delivery circuitry 206 to cardiac tissue, via the leads 219. Also not shown in FIG. 2 is the protection circuitry commonly included in implanted devices to protect, for example, the sensing circuitry of the device from high voltage stimulating pulses.

As previously noted, the primary circuitry 202 includes the processing unit 212 which generally varies in sophistication and complexity depending upon the type and functional features of the IMD 200. As described in more detail below, IMD 200 may include a suitably configured electronic control module for one or more variable capacitor elements, and the electronic control module may be realized in or executed by processing unit 212, memory unit 210, and/or elsewhere in IMD 200.

Although specific connections between the processing unit 212 and other components of the IMD 200 are not shown in FIG. 2, it will be apparent to those of ordinary skill in the art that the processing unit 212 functions to control the timed operation of the sense amplifier circuitry 204 and the therapy delivery circuitry 206. In certain embodiments, the functioning of the processing unit 212 would be under control of firmware and programmed software algorithms stored in memory 210 (e.g., RAM, ROM, PROM and/or reprogrammable ROM) and are carried out using a processing unit of a typical microprocessor core architecture. In certain embodiments, the processing unit 212 can also include a watchdog circuit, a DMA controller, a lock mover/reader, a CRC calculator, and other specific logic circuitry coupled together by on-chip bus, address bus, and power, clock, and control signal lines in paths or trees in a manner well known in the art.

In certain embodiments, as is known in the art, the electrical energy source 214 powers the primary circuitry 202 and can also be used to power electromechanical devices, such as valves or pumps, of a substance delivery IMD, or to provide electrical stimulation energy of an ICD pulse generator, cardiac pacing pulse generator, or other electrical stimulation generator. In certain embodiments, the electrical energy source 214 is a high energy density, low voltage battery coupled with a power supply circuit having power-on-reset (POR) capability. The power supply circuit provides one or more low voltage power supply signals, the POR signal, one or more voltage reference sources, current sources, an elective replacement indicator (ERI) signal, and, in the case of an ICD, high voltage power to the therapy delivery circuitry 206. For the sake of clarity in the example block diagram provided in FIG. 2, the connections between the electrical energy source 214 and the electrical components of the IMD 200 are not shown, as one skilled in the art would be familiar with such connections.

In certain embodiments, the sense amplifier circuitry 204 can be configured to process physiologic signals that are used to trigger or modulate therapy delivery and are stored as physiologic signal data for later retrieval as described herein. Generally, the sense amplifier circuitry 204 is coupled to electrical signal sense electrodes and/or physiologic sensors on or in the housing of the IMD 200 or as mentioned above, situated at sites distanced from the IMD housing, typically in distal portions of the elongated leads 219. As is generally known, the sensors or electrodes located outside the housing are coupled by conductors to feedthrough pins of feedthroughs extending through the housing wall. Certain physiologic sensors or sense electrodes can be mounted to a connector assembly so that the conductors are quite short.

In certain embodiments, the conductors include the elongated conductors of the leads 219 extending to the remotely situated physiologic sensors and sense electrodes. As such, in some cardiac applications, the sense amplifier circuitry 204 is designed to receive electrical cardiac signals from the leads 219 and to process such signals to derive event signals reflecting the occurrence of specific cardiac electrical events, including atrial contractions (P-waves) and ventricular contractions (R-waves). These event-indicating signals are provided to the processing unit 212 for use in controlling the synchronous stimulating operations of the IMD 200 in accordance with common practice in the art. In addition, these event indicating signals may be communicated, via uplink transmission, to one or more external communication devices.

In example embodiments, the therapy delivery circuitry 206 can be configured to deliver electrical stimulation to the patient, e.g., cardioversion/defibrillation therapy pulses and/or cardiac pacing pulses delivered to the heart, or other electrical stimulation delivered to the brain, other organs, selected nerves, the spinal column, the cochlea, or muscle groups, including skeletal muscle wrapped about the heart. Alternatively, in certain embodiments, the therapy delivery circuitry 206 can be configured as a drug pump delivering drugs into organs for therapeutic treatment or into the spinal column for pain relief. Alternatively, in certain embodiments, the therapy delivery circuitry 206 can be configured to operate an implantable heart assist device or pump implanted in patients awaiting a heart transplant operation.

When the IMD 200 is used for cardiac applications, the sense amplifier circuitry 204 may also include patient activity sensors or other physiologic sensors for sensing the need for cardiac output and modulating pacing parameters accordingly through many alternative approaches set forth in the prior art. If the IMD 200 is an ICD, the therapy delivery circuitry 206 generally includes one or more high power cardioversion/defibrillation output capacitors, electronic circuitry coupled to the sense amplifiers for detecting and discriminating pathologic and/or nonpathologic arrhythmias from one another and providing other functions, high voltage electronic circuitry for charging the output capacitor(s) from a battery voltage to a higher voltage, and electronic switching circuitry for dumping the charge built up on the output capacitor(s) through the cardioversion/defibrillation electrodes operatively coupled to the one or more endocardial leads 219.

Registers of the memory 210 can be used for storing data compiled from sensed cardiac activity and/or relating to device operating history or sensed physiologic parameters. Generally, the data storage can be triggered manually by the patient, on a periodic basis, or by detection logic (e.g., within the sense amplifier circuitry 204) upon satisfaction of certain programmed-in event detection criteria. If not manually triggered, in certain embodiments, the criteria for triggering data storage within the IMD 200 is programmed via telemetry transmitted instructions and parameter values. If manually triggered, in some cases, the IMD 200 includes a magnetic field sensitive switch (this may be a Hall effect sensor, or another received communications signal) that closes in response to a magnetic field, and the closure causes a magnetic switch circuit to issue a switch closed signal to the processing unit 212 which responds in a “magnet mode.” For example, the patient may be provided with a magnet (e.g., incorporated into an external communication device) that can be applied over the IMD 200 to close the switch and prompt the processing unit 212 to store physiologic episode data when the patient experiences certain symptoms and/or deliver a therapy to the patient. Following such triggering, in certain embodiments, event related data, e.g., the date and time, may be stored along with the stored periodically collected or patient initiated physiologic data. Typically, once stored, the data is ready for telemetry transmission on receipt of a retrieval or interrogation instruction.

Memory 210 may also be used to store data necessary to support the variable capacitor adjustment and programming procedures described herein. For example, memory 210 may be configured to store information related to pre-programmed capacitance settings for different operating modes of IMD 200. Memory 210 may also be configured to maintain a record of capacitance settings if so desired for diagnostic or historical tracking purposes.

In certain embodiments, the crystal oscillator circuit 208 generally employs clocked CMOS digital logic ICs having a clock signal provided by a crystal (e.g., piezoelectric) and a system clock coupled thereto as well as discrete components, e.g., inductors, capacitors, transformers, high voltage protection diodes, and the like that are mounted with the ICs to one or more substrate or printed circuit board. Typically, each clock signal generated by the system clock is routed to all applicable clocked logic via a clock tree. In certain embodiments, the system clock provides one or more fixed frequency clock signals that are independent of the battery voltage over an operating battery voltage range for system timing and control functions and in formatting telemetry signal transmissions. Again, the lines over which such clocking signals are provided to the various timed components of the IMD 200 (e.g., processing unit 212) are omitted from FIG. 2 for the sake of clarity.

Those of ordinary skill in the art will appreciate that IMD 200 may include numerous other components and subsystems, for example, activity sensors and associated circuitry. The presence or absence of such additional components in IMD 200, however, is not believed to be pertinent to the present invention, which relates to the implementation and operation of an embodiment of an input filter circuit in IMD 200, and associated techniques and technologies.

In certain embodiments, IMD 200 can involve an implantable cardiac monitor without therapy delivery system 206, e.g., an implantable EGM monitor for recording the cardiac electrogram from electrodes remote from the heart. Alternatively, IMD 200 can involve an implantable hemodynamic monitor (IHM) for recording cardiac electrogram and other physiologic sensor derived signals, e.g., one or more of blood pressure, blood gases, temperature, electrical impedance of the heart and/or chest, and patient activity.

As described above, IMD 200 includes communication module 216 and one or more antennas 218. Communication module 216 may include any number of transmitters, any number of receivers, and/or any number of transceivers, depending upon the particular implementation. In certain embodiments, each of the antennas 218 is mounted to the IMD 200 in one or more of a wide variety of configurations. For example, one or more of the antennas 218 can take the form of a surface mounted antenna, or one or more of the antennas 218 can be enclosed within or mounted to the IMD connector block assembly.

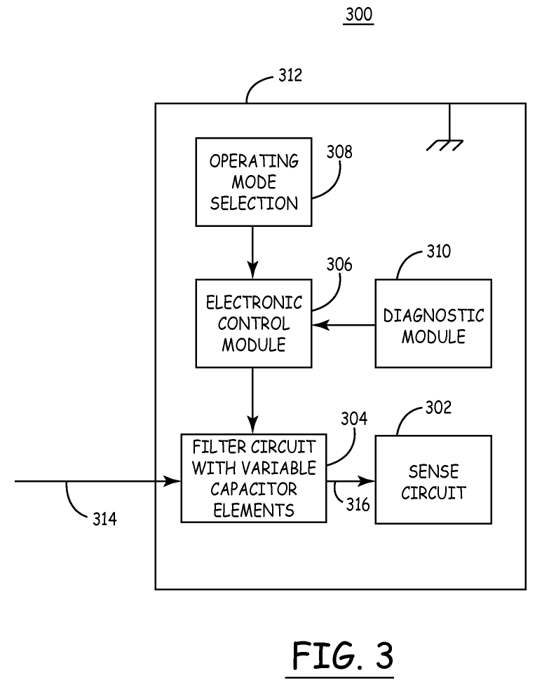

FIG. 3 is a schematic representation of an embodiment of an IMD 300 having a filter circuit with variable capacitor elements. IMD 100 and/or IMD 200 may also be suitably configured as shown in FIG. 3. FIG. 3 generally depicts (in block diagram format) a number of functional elements, circuits, and modules utilized by IMD 300. For example, IMD 300 may include, without limitation: a sense circuit 302; a filter circuit 304 coupled to sense circuit 302; and an electronic control module 306 coupled to filter circuit 304. IMD 300 may also include operating mode selection logic 308 for electronic control module 306 and/or a diagnostic module 310 coupled to electronic control module 306. These functional elements, circuits, and modules are preferably enclosed within a case or a housing 312 of IMD 300. In this embodiment, housing 312 is electrically conductive and serves as a reference potential for IMD 300 (as depicted by the chassis ground icon in FIG. 3).

Sense circuit 302 is suitably configured to process sensor signals received by IMD 300. Such sensor signals may be fed into IMD 300 via one or more electrode leads (described above). Sense circuit 302 may have any number of input nodes for a respective number of sensor signals. Filter circuit 304 is configured to filter noise from the received sensor signals using known electronic filtering techniques and topologies. For this embodiment, filter circuit 304 receives raw sensor signals 314 carried by the electrode leads, filters the raw sensor signals 314, and makes the filtered sensor signals 316 available as inputs to sense circuit 302. Filter circuit 304 includes at least one variable capacitor element that can be electrically adjusted/programmed with electronic control module 306. Referring to FIG. 2, filter circuit 304 (or portions thereof) may be realized in lead interface 220 and/or in sense amplifier circuitry 204. Similarly, sense circuit 302 (or portions thereof) may be realized in lead interface 220 and/or in sense amplifier circuitry 204.

Electronic control module 306 represents hardware, software, a state machine, and/or firmware that is suitably configured to adjust capacitance of the variable capacitor element(s) of filter circuit 304. In one embodiment, the variable capacitor elements are digitally programmable and electronic control module 306 is realized as a digital controller that generates digital control signals for programming/adjusting the variable capacitor elements. Depending upon the particular implementation of IMD 300, electronic control module 306 may be configured to: adjust the capacitance to accommodate varying operating conditions of IMD 300; dynamically adjust the capacitance in accordance with electromagnetic conditions detected by IMD 300; adjust the capacitance in accordance with a selected operating mode of IMD 300; and/or dynamically adjust the capacitance in accordance with noise conditions detected by IMD 300.

Operating mode selection logic 308 may be utilized to switch the operating mode of IMD 300. Mode switching may be initiated automatically by IMD 300, it may be initiated manually by the patient or a caregiver, or it may be initiated remotely using a wireless programmer. The selected operating mode may in turn influence the operation of electronic control module 306, which in turn may adjust the variable capacitors to tune filter circuit 304. Referring to FIG. 2, operating mode selection logic 308 (or portions thereof) may be realized in processing unit 212. Non-limiting examples of different operating modes include: a normal or default operating mode; an MRI operating mode that is selected when the patient undergoes an MRI; a hospital/clinical operating mode that is selected when the patient enters a hospital or undergoes a medical procedure in the presence of known electromagnetic energy sources; and modes for procedures such as electrocautery, CT scans, lithotripsy, RF ablation, electrolysis, and diathermy.

Diagnostic module 310 represents hardware, software, a state machine, and/or firmware that is suitably configured to detect electromagnetic or noise conditions for IMD 300. Referring to FIG. 2, diagnostic module 310 (or portions thereof) may be realized in processing unit 212. Current operating conditions detected by diagnostic module 310 can be provided to electronic control module 306, which in turn can adjust the capacitance of the variable capacitor elements in a desired manner in response to the detected operating conditions. For example, in the presence of strong gradient magnetic fields (such as may be experienced during an MRI), the capacitances in filter circuit 304 can be reduced in an attempt to dissipate coupled signals on the electrode leads. On the other hand, if electrical interference sources are present, then the capacitances in filter circuit 304 can be increased.

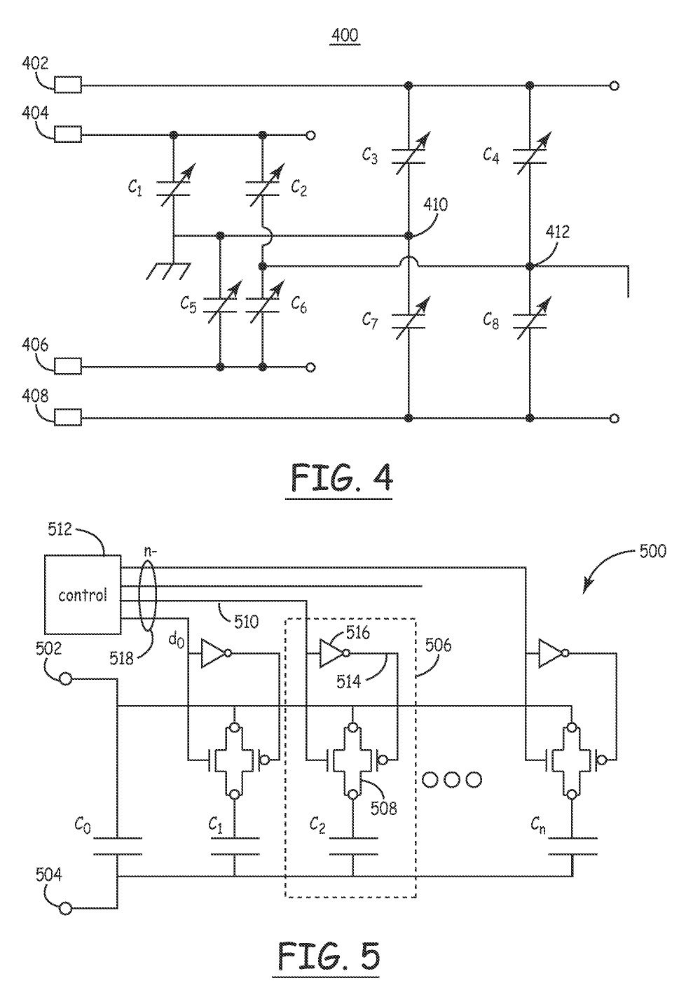

FIG. 4 is a schematic diagram of an embodiment of a filter circuit 400 having variable capacitor elements. Filter circuit 400 is one possible topology for filter circuit 304 in IMD 300. In practice, filter circuit 304 may be configured differently depending upon the number and type of electrode leads, the desired filtering characteristics, the configuration of sense circuit 302, and the like. The layout of filter circuit 400 is not intended to limit or otherwise restrict the scope or application of the embodiments described herein.

Filter circuit 400 is suitable for use with two differential bipolar electrode leads (each having two connectors). Accordingly, filter circuit 400 is depicted with four input nodes (reference numbers 402, 404, 406, and 408) for respective sensor signals. These input nodes may also represent the inputs to a sense circuit; the open circles in FIG. 4 may serve as contact points for the sense circuit. Filter circuit 400 also includes a first reference node 410 that corresponds to a first reference potential, and a second reference node 412 that corresponds to a second reference potential. Here, reference node 410 is coupled to an electrically conductive housing of the IMD and the first reference potential corresponds to the chassis ground, while reference node 412 corresponds to a ground potential for the sense circuit and/or filter circuit 400.

Filter circuit 400 includes eight capacitor elements (labeled C1 to C8). Although not a requirement, each capacitor element is configured as a variable capacitor element in this embodiment, and each variable capacitor element is coupled between one of the input nodes and one of the reference nodes. For this example, C1 is connected between input node 404 and reference node 410, C2 is connected between input node 404 and reference node 412, C3 is connected between input node 402 and reference node 410, C4 is connected between input node 402 and reference node 412, C5 is connected between input node 406 and reference node 410, C6 is connected between input node 406 and reference node 412, C7 is connected between input node 408 and reference node 410, and C8 is connected between input node 408 and reference node 412. Under normal operating conditions for an exemplary IMD implementation, C1, C3, C5, and C7 may each have a capacitance of about 1.5 nF, and C2, C4, C6, and C8 may each have a capacitance of about 3.3 nF. Of course, different nominal capacitance values may be utilized in an embodiment of filter circuit 400. Although not a requirement or limitation of the embodiments of the invention, a general adjustment range of 1.0 nF to 10.0 nF would be suitable for typical applications.

As mentioned previously, control signals generated by the IMD itself are preferably utilized to adjust or program each of the variable capacitor elements in filter circuit 400. Although not shown in FIG. 4, a suitably configured electronic control module may be utilized to tune each of the variable capacitor elements. In this regard, FIG. 5 is a schematic diagram of an embodiment of a variable capacitor element 500 that is suitable for use in a filter circuit such as filter circuit 400. Each of the variable capacitor elements C1-C8 in FIG. 4 may be configured as shown in FIG. 5.

Variable capacitor element 500 is coupled between two nodes 502/504, where the total capacitance of variable capacitor element 500 is measured between nodes 502/504. Referring to FIG. 4, node 502 may be an input node for a sensor signal and node 504 may be a reference node (or vice versa). Although not required, variable capacitor element 500 includes a fixed capacitor (C₀) that represents a minimum capacitance for variable capacitor element 500. As shown in FIG. 5, C₀ is coupled between nodes 502/504. Variable capacitor element 500 also includes at least one switched capacitance coupled between nodes 502/504, where each switched capacitance includes a switch in series with a capacitor. FIG. 5 depicts a generalized embodiment where a plurality of switched capacitances are coupled in parallel between nodes 502/504.

The dashed lines in FIG. 5 indicate one of the switched capacitances 506. Switched capacitance 506, which is representative of all switched capacitances in variable capacitor element 500, includes a fixed capacitor (C₂) in series with an electrically-gated transistor-based switch 508. Switch 508 is electrically controlled to either insert capacitor C₂ between nodes 502/504 or disconnect capacitor C₂ from the parallel combination with capacitor C₀. An enable/disable control signal 510 is delivered to switch 508 from an electronic control module 512 (as described above in the context of FIG. 3); control signal 510 controls the state of switch 508. In this manifestation of switch 508, a complementary parallel combination of an NMOS and PMOS device receives control signal 510 and its complement 514, which is generated by an inverter 516. The resistance of the electrical gate can be selected via appropriate transistor design and sizing. Alternatively, switch 508 may be realized as any electrically enabled, low impedance switch such as a solid state relay, a semiconductor-controlled switch, or a semiconductor-controlled rectifier.

Again, fixed capacitor C₀ represents the nominal baseline capacitance for variable capacitor element 500. This value may be selected such that a failure in any of the switched capacitances or in the control circuitry will still allow the IMD to be operated safely under normal use conditions. The switched capacitances are also coupled between nodes 502/504, and electronic control module 512 is suitably configured to control the switch for each of the switched capacitances. The number of switched capacitances can be determined by design requirements including, without limitation: the desired capacitance range; electrical power consumption; mechanical requirements; and space limitations. Each of the switched capacitances can be independently switched on or off via digital control logic that generates digital control signals 518 in an appropriate manner. When a particular switched capacitance is on, the respective series capacitor contributes its capacitance to the node in parallel with fixed capacitor C₀ and any other switched capacitances that may be on.

The total capacitance between nodes 502/504 can be calculated via a simplified equation such as

$${C}_{total} = {C}_{0} + {\sum }_{i = 1}^{n} {d}_{i} \cdot {C}_{i} ,$$

where dᵢ is the input control signal (1=ON, 0=OFF), n is the number of switched capacitance legs, and Cᵢ is the value of the capacitance for each leg. In addition, the parasitics of the switches may need to be considered in the design of a practical filter circuit. Given the desired frequency contents of the signal (less than 100 Hz) and the typical values of input capacitance (in the low nF range), as long as the resistive contribution of the gated switch structure is less than several hundred ohms, the electrical impact of the switches is negligible. Design requirements on the resistance of the switches can be altered depending on the application.

The digital control signals 518 can be pre-programmed for certain device conditions (e.g., MRI-safe programming mode) or they can be dynamically switched by the device to adjust to various environmental conditions. The sizes of the fixed capacitors can also be chosen to best reflect or anticipate expected device conditions or modes.

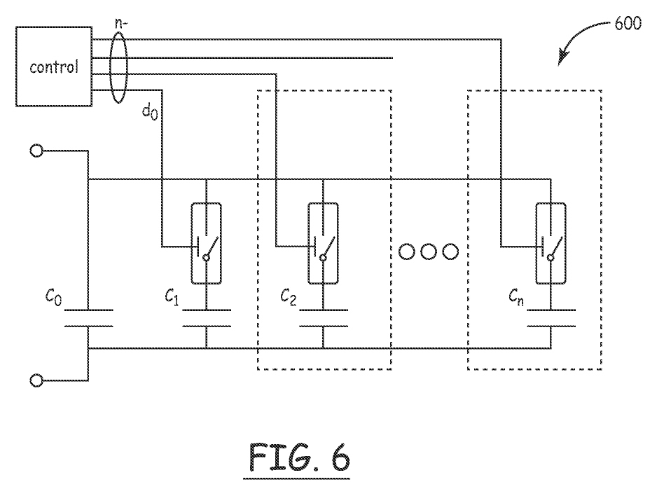

FIG. 6 is a schematic diagram of another embodiment of a variable capacitor element 600 that is suitable for use in a filter circuit such as filter circuit 400. Each of the variable capacitor elements C1-C8 in FIG. 4 may be configured as shown in FIG. 6. Variable capacitor element 600 is similar to variable capacitor element 500, and common features and functionality will not be redundantly described here. In lieu of transistor-based gated switches, variable capacitor element 600 utilizes solid-state relays that serve as the switches for the parallel switched capacitances.

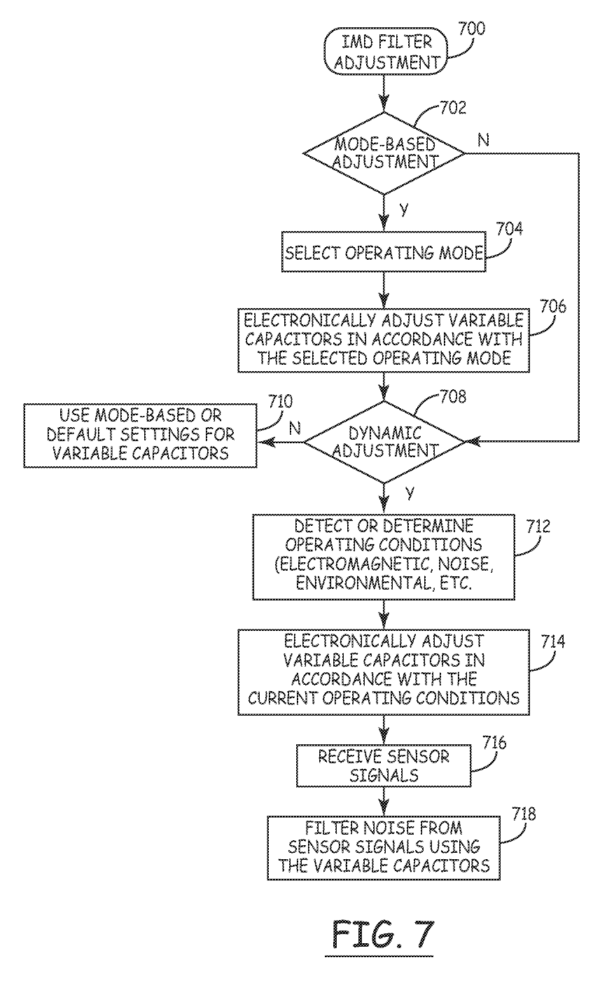

FIG. 7 is a flow chart that illustrates an embodiment of an IMD filter adjustment process 700 that may be performed by an IMD having a tunable input filter circuit. The various tasks performed in connection with process 700 may be performed by software, hardware, firmware, or any combination thereof. For illustrative purposes, the following description of process 700 may refer to elements mentioned above in connection with FIGS. 1-6. Portions of process 700 may be performed by different elements of the described system, e.g., a control module, a processor, a filter circuit, or a variable capacitor element. It should be appreciated that process 700 may include any number of additional or alternative tasks, the tasks shown in FIG. 7 need not be performed in the illustrated order, and process 700 may be incorporated into a more comprehensive procedure or process having additional functionality not described in detail herein.

For the sake of completeness, this embodiment of IMD filter adjustment process 700 assumes that the IMD supports both mode-based adjustments (that are responsive to the switching of IMD operating modes) and dynamic adjustments (that are responsive to changing operating conditions). In practice, an IMD may be configured to support only one of these adjustment methodologies.

In this example, IMD filter adjustment process 700 checks whether a mode-based adjustment has been requested (query task 702). A mode switching request may be generated and processed internally by the IMD, it may be initiated by the patient or a caregiver, it may be initiated by a programmer of the IMD, or the like. If no mode-based adjustment has been requested, then process 700 may exit or it may proceed to a query task 708 (as depicted in FIG. 7). If a mode-based adjustment was requested, then process 700 may select a current operating mode from a plurality of designated operating modes for the IMD (task 704). As mentioned above, the IMD may support different operating modes that correspond to different operating, environmental, and/or noise conditions (for example, a normal mode, an MRI mode, a surgery mode, etc.). Thereafter, process 700 electronically adjusts the capacitance of the variable capacitor element(s) in accordance with the selected and current operating mode (task 706).

This embodiment of IMD filter adjustment process 700 also checks whether it is appropriate to perform dynamic adjustments of the IMD filter circuit (query task 708). If not, then process 700 may cause the IMD to use the mode-based capacitance settings or default capacitance settings for the variable capacitor(s) (task 710). In this regard, default capacitance settings may correspond to nominal capacitance values as electronically controlled by the IMD in the absence of other adjustment instructions. If dynamic adjustments are supported, then process 700 may detect or determine (task 712) one or more conditions for the IMD (e.g., electromagnetic conditions, noise conditions, interference conditions, environmental conditions, or other conditions that might otherwise influence the signal sensing performance of the IMD). Thereafter, process 700 electronically adjusts the capacitance of the variable capacitor element(s) in accordance with the detected and current operating conditions (task 714).

An embodiment of an IMD may utilize digitally programmable variable capacitor elements that can be adjusted/programmed using digital control signals generated by suitably configured digital control logic. In such an embodiment, the various electronic adjustment tasks described herein may be accomplished by generating digital control signals for the variable capacitor elements and controlling the capacitance of each variable capacitor element in response to the digital control signals. As described above with reference to FIG. 5 and FIG. 6, a variable capacitor element may include a plurality of capacitors and adjustment may involve the selection of one or more of the plurality of capacitors for coupling in parallel between the desired nodes (e.g., the input and reference nodes of the filter circuit). This selection and parallel coupling may be realized using the switched capacitor arrangements described herein.

Once the capacitances in the filter circuit have been adjusted by the desired amount, the IMD can receive and obtain sensor signals (task 716) as inputs. In addition, the IMD can filter noise from the sensor signals using the variable capacitor element(s) of the input filter circuit (task 718). Following task 718, IMD filter adjustment process 700 may exit in an appropriate manner. For example, process 700 may return to query task 702 to monitor for a change in the current operating mode, or process 700 may return to query task 708 to facilitate dynamic updating of the variable capacitor element(s). This enables the IMD to improve its sensor signal processing quality in the presence of different levels of noise or interference.

While at least one example embodiment has been presented in the foregoing detailed description, it should be appreciated that a vast number of variations exist. It should also be appreciated that the example embodiment or embodiments described herein are not intended to limit the scope, applicability, or configuration of the invention in any way. Rather, the foregoing detailed description will provide those skilled in the art with a convenient road map for implementing the described embodiment or embodiments. It should be understood that various changes can be made in the function and arrangement of elements without departing from the scope of the invention, where the scope of the invention is defined by the claims, which includes known equivalents and foreseeable equivalents at the time of filing this patent application.

What is claimed is:

1. A filter circuit for a sense circuit of an implantable medical device, the filter circuit comprising:
an input node for a sensor signal;
a reference node corresponding to a reference potential for the implantable medical device;
a variable capacitor element coupled between the input node and the reference node; and
an electronic control module coupled to the variable capacitor element, the electronic control module being configured to adjust capacitance of the variable capacitor element to accommodate varying operating conditions of the implantable medical device.

2. A filter circuit according to claim 1, wherein the reference node is coupled to a conductive housing of the implantable medical device.

3. A filter circuit according to claim 1, wherein the reference node corresponds to a ground potential for the sense circuit.

4. A filter circuit according to claim 1, wherein:
the variable capacitor element comprises at least one switched capacitance having a capacitor in series with a switch; and
the electronic control module controls the switch.

5. A filter circuit according to claim 1, wherein the variable capacitor element comprises:
a fixed capacitor coupled between the input node and the reference node; and
a switched capacitance coupled between the input node and the reference node, the switched capacitance comprising a switch in series with a capacitor; wherein
the electronic control module controls the switch.

6. A filter circuit according to claim 1, wherein the electronic control module is configured to dynamically adjust capacitance of the variable capacitor element in accordance with electromagnetic conditions detected by the implantable medical device.

7. A filter circuit according to claim 1, wherein the electronic control module is configured to adjust capacitance of the variable capacitor element in accordance with a selected operating mode of the implantable medical device.

8. An implantable medical device comprising:
a sense circuit configured to process a sensor signal, the sense circuit having an input node for the sensor signal;
a reference node corresponding to a reference potential for the implantable medical device;
a filter circuit configured to filter noise from the sensor signal, the filter circuit comprising a variable capacitor element coupled between the input node and the reference node; and
an electronic control module for the variable capacitor element, the electronic control module being configured to adjust capacitance of the variable capacitor element.

9. An implantable medical device according to claim 8, further comprising a conductive housing enclosing the sense circuit, the filter circuit, and the electronic control module, wherein the reference node is coupled to the conductive housing.

10. An implantable medical device according to claim 8, wherein the reference node corresponds to a ground potential for the filter circuit.

11. An implantable medical device according to claim 8, wherein:
the variable capacitor element is digitally programmable; and
the electronic control module is configured to generate digital control signals for programming the variable capacitor element.

12. An implantable medical device according to claim 8, wherein:
the variable capacitor element comprises a plurality of switched capacitances coupled in parallel between the input node and the reference node;
each of the switched capacitances comprises a switch in series with a capacitor; and
the electronic control module controls the switch for each of the switched capacitances.

13. An implantable medical device according to claim 8, further comprising a diagnostic module coupled to the electronic control module, the diagnostic module being configured to detect noise conditions for the implantable medical device, wherein the electronic control module is configured to dynamically adjust capacitance of the variable capacitor element in accordance with the noise conditions detected by the diagnostic module.

14. An implantable medical device according to claim 8, wherein the electronic control module is configured to adjust capacitance of the variable capacitor element in accordance with a selected operating mode of the implantable medical device.

15. A method of operating an implantable medical device having an input node, a reference node corresponding to a reference potential, and a variable capacitor element coupled between the input node and the reference node, the method comprising:
electronically adjusting capacitance of the variable capacitor element in accordance with an operating condition of the implantable medical device;
obtaining a sensor signal at the input node; and
filtering noise from the sensor signal using the variable capacitor element.

16. A method according to claim 15, further comprising detecting electromagnetic conditions for the implantable medical device, wherein the adjusting step dynamically adjusts capacitance of the variable capacitor element in accordance with the electromagnetic conditions.

17. A method according to claim 15, further comprising selecting a current operating mode from a plurality of designated operating modes for the implantable medical device, wherein the adjusting step adjusts capacitance of the variable capacitor element in accordance with the current operating mode.

18. A method according to claim 15, wherein:
the variable capacitor element is digitally programmable;
the method further comprises generating digital control signals for programming the variable capacitor element; and
the adjusting step adjusts capacitance of the variable capacitor element in response to the digital control signals.

19. A method according to claim 15, wherein:
the variable capacitor element comprises a plurality of capacitors; and
the adjusting step comprises selecting one or more of the plurality of capacitors to obtain selected capacitors, and coupling the selected capacitors in parallel between the input node and the reference node.
